(19) United States
(12) Patent Application Publication
(10) Pub. No.: US 2023/0076298 A1
(43) Pub. Date: Mar. 9, 2023

(54) CONSENSUS BUILDING SUPPORT SYSTEM, APPARATUS, AND METHOD

(71) Applicant: Canon Medical Systems Corporation, Otawara-shi (JP)

(72) Inventors: Anri YAMAZAKI, Nasushiobara (JP); Yusuke KANO, Nasushiobara (JP); Minoru NAKATSUGAWA, Yokohama (JP)

(73) Assignee: Canon Medical Systems Corporation, Otawara-shi (JP)

(21) Appl. No.: 17/883,673

(22) Filed: Aug. 9, 2022

(30) Foreign Application Priority Data
Sep. 3, 2021 (JP) 2021-143849

(51) Int. Cl. G16H 15/00; G16H 50/20; G16H 40/63; G16H 10/60

(52) U.S. Cl. CPC G16H 15/00; G16H 50/20; G16H 40/63; G16H 10/60

(57) ABSTRACT

A consensus building support system according to an embodiment includes processing circuitry. The processing circuitry stores a database that stores a state of a first user and actions by the first user and a second user. The processing circuitry calculates a first state assessment index representing a first user's assessment of the state based on a first user's state and an action relating to the first user, and calculates a second state assessment index representing a second user's assessment of the state based on the first user's state and an action relating to the second user. The processing circuitry display a first state assessment index and/or a second state assessment index on a display.

### CROSS-REFERENCE TO RELATED APPLICATIONS

This application is based upon and claims the benefit of priority from Japanese Patent Application No. 2021-143849, filed Sep. 3, 2021, the entire contents of which are incorporated herein by reference.

### FIELD

Embodiments described herein relate generally to a consensus building support system, apparatus, and method.

### BACKGROUND

Shared decision making in which a clinician and a patient share a decision-making process and a rationale and results of decision making is important in order to encourage the patient to understand their own illness, to agree with a treatment plan, and to continue to participate in treatment.

### BRIEF DESCRIPTION OF THE DRAWINGS

FIG. 1 is a diagram showing a configuration example of a consensus building support system according to a first embodiment.

FIG. 2 is a diagram showing a configuration example of a consensus building support apparatus according to a first embodiment.

FIG. 3 shows an example of a consensus building support process by the consensus building support apparatus.

FIG. 4 is a diagram showing an example of an administration record of an instruction.

FIG. 5 is a diagram showing an example of a nursing record.

FIG. 6 is a diagram showing examples of states and combined actions.

FIG. 7 is a diagram showing examples of agents of combined actions.

FIG. 8 is a diagram showing relationships between states s(t), states s(t+1), and actions a_{dr}(t) associated with a doctor from those listed in FIG. 7.

FIG. 9 is a diagram showing a relationship between states s(t), states s(t+1), and actions aₚₜ(t) associated with a patient from those listed in FIG. 7.

FIG. 10 is a diagram showing an example of a display screen of a patient's state assessment index map and a doctor's state assessment index map.

FIG. 11 is a diagram showing an example of a display screen of a patient's state assessment index map and a doctor's state assessment index map.

FIG. 12 is a diagram showing an example of a display screen of a change over time of a state combination corresponding to a maximum value of a state assessment index.

FIG. 13 is a diagram showing a configuration example of a consensus building support apparatus according to a second embodiment.

FIG. 14 is a diagram showing an example of a display screen of a graph representing a change over time of a difference value between the patient's state assessment index and the doctor's state assessment index.

FIG. 15 is a diagram showing an example of a display window for a recommended action candidate.

FIG. 16 is a diagram showing an example of a display window of bases for calculating a recommended action candidate.

FIG. 17 is diagrams showing a path peripheral area of an optimal action path of a patient and that of an optimal action path of a doctor.

FIG. 18 is a diagram showing a past action path and a future action path in a state space.

### DETAILED DESCRIPTION

With the system according to Jpn. PCT National Publication No. 2017-519303, patient's preferences, reasons for choices, and a level of a patient's understanding about their illness are estimated based on patient's answers to a questionnaire. However, this method requires a lot of effort from the patient who is required to answer a questionnaire. In addition, with this system, it is difficult to tell whether the doctor understands the patient, and it is therefore a one-way shared decision-making process.

The consensus building support system according to an embodiment includes processing circuitry. The processing circuitry stores a database that stores states of a first user and actions taken by the first user and a second user. The processing circuitry calculates a first state assessment index representing a first user's assessment of the state based on a first user's state and an action relating to the first user, and calculates a second state assessment index representing a second user's assessment of the state based on the first user's state and an action relating to the second user. The processing circuitry display a first state assessment index and/or a second state assessment index on a display.

Hereinafter, embodiments of a consensus building support system, apparatus, and method will be explained in detail with reference to the accompanying drawings.

### First Embodiment

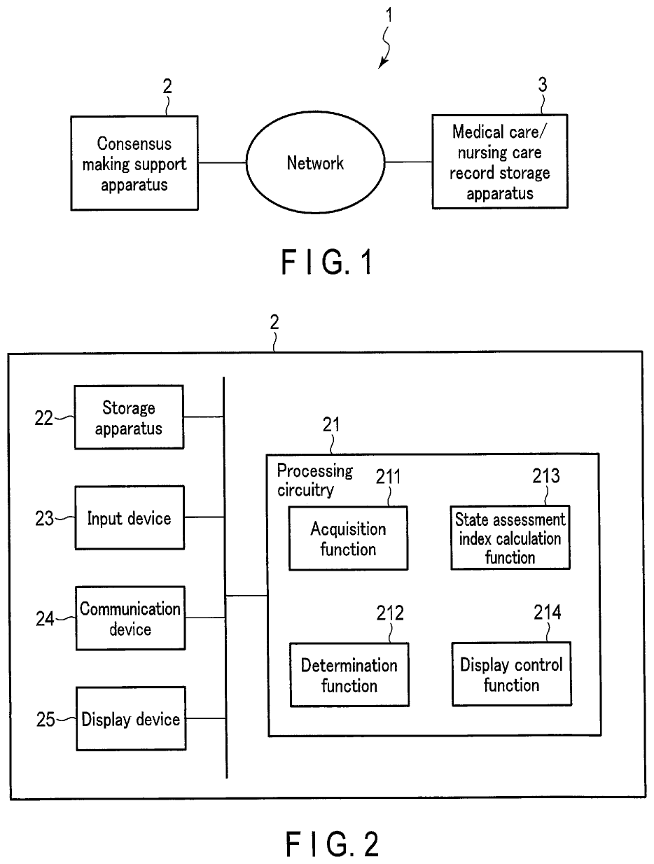

FIG. 1 is a diagram showing a configuration example of a consensus building support system 1 according to the first embodiment. As shown in FIG. 1, the consensus building support system 1 is a computer network in which a consensus building support apparatus 2 and a medical care/nursing care record storage apparatus 3 are connected to each other through a network in a communicable manner.

The consensus building support apparatus 2 is a computer configured to aid consensus building between users regarding medical care and/or nursing care. Specifically, the consensus building support apparatus 2 acquires medical care records and/or nursing care records from the medical care/nursing care record storage apparatus 3, produces support information for consensus building based on the acquired medical care records and/or nursing care records, and display the produced support information on a display. The user according to the present embodiment includes a patient, a patient's family, a healthcare worker, a care worker, etc. A healthcare worker in the present embodiment includes a doctor, a pharmacist, and a nurse, and the like. A care worker in the present embodiment includes a care service staff, a certified care worker, a social worker, a care manager, and the like.

The medical care/nursing care record storage apparatus 3 is a computer that stores a database for medical care records and/or nursing care records of a number of patients. A medical care record in the present embodiment is a chronological record of patient's states and actions taken by a user (patient, family, healthcare worker, etc.) for the state and produced in the process of giving medical care to a patient. A nursing care record in the present embodiment is a chronological record of patient's states and actions taken by a user (patient, family, care worker, etc.) for the state and produced in the process of giving nursing care.

FIG. 2 is a diagram showing a configuration example of a consensus building support apparatus 2 according to the first embodiment. As shown in FIG. 2, the consensus building support apparatus 2 has processing circuitry 21, a storage apparatus 22, an input device 23, a communication device 24, and a display device 25.

The processing circuitry 21 includes processors such as a CPU (central processing unit) and a GPU (graphics processing unit). The processing circuitry 21 executes a consensus building support program to implement an acquisition function 211, a determination function 212, a state assessment index calculation function 213, and a display control function 214. Note that the embodiment is not limited to the case in which the respective functions 211 to 214 are implemented by single processing circuitry. Processing circuitry may be composed by combining a plurality of independent processors, and the respective processors may execute programs, thereby realizing the functions 211 to 214. The functions 211 to 214 may be respective modularized programs constituting a consensus building support program or separate programs. These programs are stored in the storage apparatus 22.

Through implementation of the acquisition function 211, the processing circuitry 21 acquires various information items. For example, the processing circuitry 21 acquires a first user's state and first and second users' actions from the database of the medical care/nursing care record storage apparatus 3. The first user is a user targeted for processing and an agent of a state. The first user is typically a patient. The second user is a user other than a user targeted for processing, for example, a patient's family, a healthcare worker, or a nursing care worker. In other words, the second user is a person who builds a consensus together with the first user.

Through implementation of the determination function 212, the processing circuitry 21 determines that the agent of an action acquired by the acquisition function 211 is a first user or a second user.

Through implementation of the state assessment index calculation function 213, the processing circuitry 21 calculates a first state assessment index representing a first user's assessment of a state based on a first user's state and an action relating to the first user. The processing circuitry 21 calculates a second state assessment index representing a second user's assessment of a state based on a first user's state and an action relating to the second user. The first state assessment index and the second state assessment index are an example of support information for consensus building.

Through implementation of the display control function 214, the processing circuitry 21 displays various information items on the display device 25. For example, the processing circuitry 21 displays the first state assessment index and/or the second state assessment index on the display device 25.

The storage apparatus 22 is a type of storage such as a ROM (read only memory), a RAM (random access memory), an HDD (Hard Disk Drive), an SSD (Solid State Drive), or an integrated circuit storage device, etc. which stores various types of information. The storage apparatus 22 may be not only the above-listed memory apparatuses, but also a driver that writes and reads various types of information to and from, for example, a portable storage medium such as a compact disc (CD), a digital versatile disc (DVD), or a flash memory, or a semiconductor memory. The storage apparatus 22 may be provided in another computer connected via a network.

The input device 23 accepts various kinds of input operations from an operator, converts the accepted input operations to electric signals, and outputs the electric signals to the processing circuitry 21. Specifically, the input device 23 is connected to an input device, such as a mouse, a keyboard, a trackball, a switch, a button, a joystick, a touch pad, or a touch panel display. The input device 23 outputs to the processing circuitry 21 an electrical signal corresponding to an input operation on the input device. The input device 23 may be an input device provided in another computer connected via a network or the like.

The communication device 24 is an interface for transmitting and receiving various types of information with other computers such as the medical care/nursing care record storage apparatus 3 included in the consensus building support system 1.

The display device 25 displays various types of information through the display control function 214 of the processing circuitry 21. For the display device 25, for example, a liquid crystal display (LCD), a cathode ray tube (CRT) display, an organic electro luminescence display (OELD), a plasma display, or any other display can be used as appropriate. A projector may be used as the display device 25.

An example of the operation of the consensus building support apparatus 2 according to the first embodiment is described below. In the following description, assume that a first user is a patient and a second user is a doctor. Assume that the medical care/nursing care record storage apparatus 3 stores an electronic medical care record database for recording medical care records. The electronic medical care record may be called an “electronic health record”. The consensus building support apparatus 2 performs consensus building support for supporting a shared decision making between a patient and a doctor.

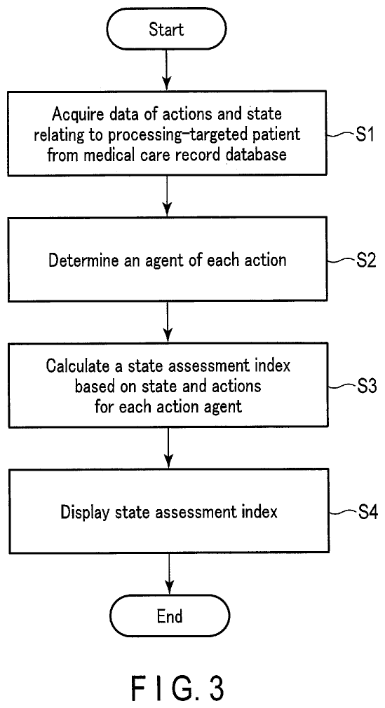

FIG. 3 shows an example of a consensus building support process by the consensus building support apparatus 2. As shown in FIG. 3, the processing circuitry 21 obtains, through the implementation of the acquisition function 211, data of an action and a state relating to a patient who is a target of processing from the medical care record database (step S1).

The “state” means one of the states of the patient in a patient's medical care. The “action” means an action taken by the patient or the doctor or by both parties for the “state” of the patient. In other words, the “state” is changeable depending on the “action” and is observable. The “state” varies in type, from vital data to an examination value, to a medical bill, a subjective symptom, a therapeutic effect, a resource, a side-effect, and the like. The state of the acquisition target in step S1 may be any type, and it suffices that at least one state is acquired. Preferably, the type of the state acquired in step S1 is determined in advance either manually or automatically. In the following examples, assume that two types of state, a presence/absence of edema and an SpO₂ value, are acquired. The state of the patient in this case will be defined by a combination of a presence/absence of edema and an SpO₂ value. Such a combination will be called a state combination.

The medical care record database includes administration records of instructions order and nursing records. In the administration records, actions actually taken by the doctor, etc. are recorded in chronological order, as an instructions order. In the nursing records, patient's states and actions taken for the states are recorded in chronological order. The actions recorded in the nursing records may be an action taken by the patient or an action taken by a healthcare worker, such as a doctor or a nurse.

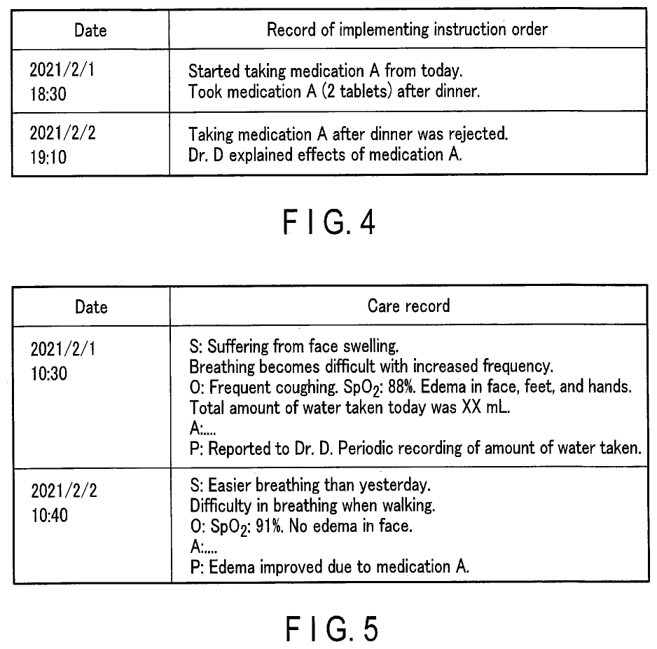

FIG. 4 is a diagram showing an example of an administration record of an instruction order. FIG. 5 is a diagram showing an example of a nursing record. As shown in FIG. 4, dates and administration of actions are recorded using letters and numbers in the administration records. For example, actions taken by a doctor are recorded in writing in the administration record shown in FIG. 4. As shown in FIG. 5, dates and nursing actions are recorded using characters and numbers in the nursing records. Specifically, SOAP notes are recorded in the nursing records in FIG. 5. A patient's subjective information is recorded in the “S” section, objective information regarding the patient's state is recorded in the “O” section, an assessment is recorded in the “A” section, and an action taken by a healthcare worker, for example a doctor, is recorded in the “P” section.

In step S1, as an example, the processing circuitry 21 acquires data of actions and states from the administration records of the instructions order and the nursing records by natural language processing. A state and an action taken for the state constitutes a combination. The action and the state are acquired and associated with a value representing the time and date of the acquisition. The action and state may be acquired in association with an identifier representing a source of acquisition. At the time of step S1, the agent of the “action” may not be necessarily determined.

The method of acquiring action and state data is not limited to natural language processing. As another example, an identifier (ID) for identifying content of each item, such as an instruction and administration order, etc. in the medical care record database may be assigned, and such an identifier may be referred to in order to acquire data of action and state. For example, an item corresponding to the identifier “1” is provided for an instruction or an administration order, and a presence/absence of an edema is recorded in the section of the item as a “state”. In step S1, the processing circuitry 21 searches for the item with the identifier “1” when a presence/absence of an edema is acquired, and acquires the presence/absence of an edema from this item.

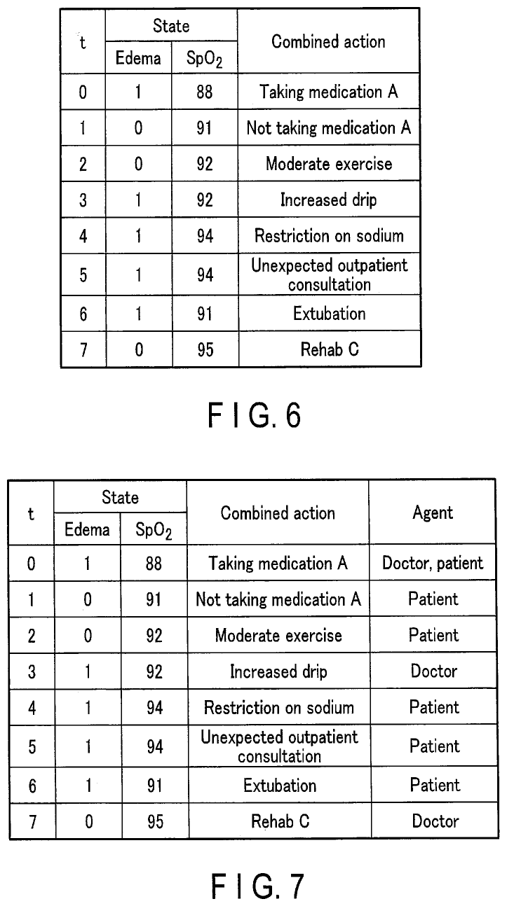

FIG. 6 is a diagram showing examples of states and combined actions. A “combined action” is an action taken by either a patient or a doctor or by both of them. As shown in FIG. 6, a combination of a state and a combined action is acquired every time (step) t. A state is a combination of a presence/absence of edema and an SpO₂ value. The presence/absence of edema is represented by a categorical value. For example, “0” represents “no edema” and “1” represents “with edema”. The conversion of a presence/absence of edema into a categorical value may be performed by the processing circuitry 21. SpO₂ is represented by a continuous value, which is a measured value itself. SpO₂ can also be converted into a categorical value in accordance with a level of a measured value. The combined action is represented by a character sequence. The combined action can also be converted into a categorical value. For example, “Taking medication A” may be converted into “0001” and “Not taking medication A” may be converted into “0002”, for example.

After step S1, the processing circuitry 21 determines an agent of each action through implementation of the determination function 212 (step S2).

FIG. 7 is a diagram showing examples of agents of combined actions. As shown in FIG. 7, for each combined action, an agent is determined to be either a patient and a doctor or is determined to be both a patient and a doctor. A method of determining an agent is automatic based on a source or content of the combined action.

An example of a determination method based on a source of a combined action is as follows. If a combined action is acquired from an instruction, the processing circuitry 21 determines that the agent of the combined action is a doctor, and if a combined action is acquired from an administration order, the processing circuitry 21 determines that the agent is a patient. As another method, if a combined action is acquired from the “P” section of a SOAP note, the processing circuitry 21 determines that the agent of the combined action is a doctor, and if from the “S” section, the agent is a patient. As another example, the processing circuitry 21 may determine the agent of a combined action by a rule-based processing based on an order ID. More specifically, if a combined action is acquired from a predetermined item included in an order, the processing circuitry 21 determines whether the agent of the combined action is a doctor or a patient in accordance with an identifier assigned to the predetermined item.

In the case of a determination method based on natural language processing, the processing circuitry 21 performs natural language processing on a character sequence including characters describing a combined action so as to determine whether the agent of the combined action is a doctor or a patient. The character sequence includes not only characters describing a combined action but also characters describing an agent of a combined action, or characters for inferring the agent. Typically, it suffices that natural language processing is performed on a character sequence describing a date of treatment in electronic medical care records.

After step S2, the processing circuitry 21 calculates a state assessment index based on a state and an action for each action agent through implementation of the state assessment index calculation function 213 (step S3). Assume that a series of states in the present example follows a Markov determination process. In this case, in step S3, the processing circuitry 21 calculates the patient's state assessment index rₚₜ by inverse reinforcement learning based on the processing-target state s(t), the next step state s(t+1), and a patient's action aₚₜ(t) between the processing-target state s(t) and the next step state s(t+1). Similarly, the processing circuitry 21 calculates the doctor's state assessment index r_{dr} by inverse reinforcement learning based on the processing-target state s(t), the next step state s(t+1), and a doctor's action a_{dr}(t) between the processing-target state s(t) and the next step state s(t+1).

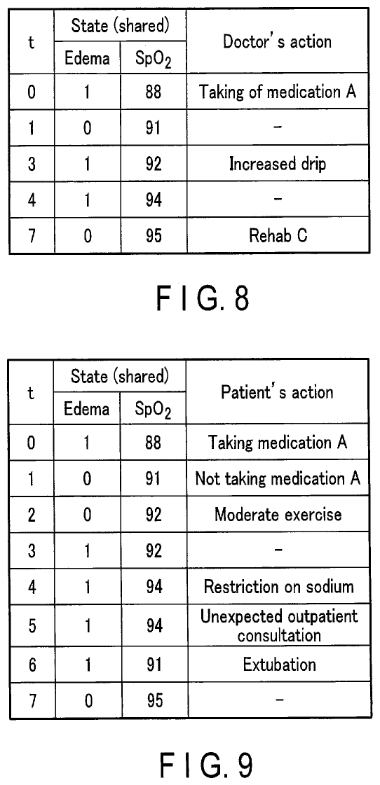

FIG. 8 is a diagram showing relationships between states s(t), states s(t+1), and actions a_{dr}(t) associated with the doctor from those listed in FIG. 7. FIG. 9 is a diagram showing a relationship between states s(t), states s(t+1), and actions aₚₜ(t) associated with the patient from those listed in FIG. 7. The state associated with the action taken by the doctor and the state associated with the action taken by the patient are both the patient's state. The state assessment indices rₚₜ(t) and r_{dr}(t) are calculated for step t where there are the actions aₚₜ(t) and a_{dr}(t). For example, as shown in FIG. 8, there is the action a_{dr}(0) when t=0, and the state assessment index r_{dr} is calculated, whereas the state assessment index r_{dr} is not calculated as there is no action a_{dr}(1) when t=1. Similarly, for example, as shown in FIG. 9, there is an action aₚₜ(0) when t=0 and therefore the state assessment index rₚₜ is calculated; however, there is no action aₚₜ(3) when t=3, and the state assessment index rₚₜ is not calculated.

Typically, the state assessment indices rₚₜ and r_{dr} are calculated as the rewards Rₚₜ(s) and R_{dr}(s) in inverse reinforcement learning. For example, if an optimal action a* in each state si is known from past findings, etc., the rewards Rₚₜ(s) and R_{dr}(s) can be calculated using a linear planning method, etc. in which a difference between a reward and an expected reward in the optimal action a* is used. If an optimal action is unknown, the rewards are calculated using Maximum Entropy inverse reinforcement learning, using a measure used in the past. In this case, it is preferable if the processing circuitry 21 converts the action aₚₜ (t) and action a_{dr}(t) written in character sequences into categorical values. The character sequence and the categorical value to describe content of an action are associated in a table, etc. in advance, and the processing circuitry 21 may convert the character sequence describing content of an action into a categorical value, using the table.

As another example, the state assessment indices rₚₜ and raᵣ may be calculated as an optimal measure n(s) in an imitation learning algorithm, etc. using inverse reinforcement learning, such as generative adversarial imitation learning (GAIL). As another example, the state assessment indices rₚₜ and R_{dr} may be calculated as the rewards Rₚₜ(s,a) and R_{dr}(s,a), the action value functions Qₚₜ(s) and Q_{dr}(s), and the state value functions Vₚₜ(s) and V_{dr}(s).

After step S3, the processing circuitry 21, through implementation of the display control function 214, displays the state assessment index calculated in step S3 (step S4). In step S4, the processing circuitry 21 display the state assessment index relating to a patient and/or the state assessment index relating to a doctor on the display device 25. As an example, the processing circuitry 21 displays a map of the state assessment index relating to a patient and/or a map of the state assessment index relating to a doctor (the maps will be called “state assessment index map”, respectively).

Generally speaking, a state is represented by a time series of combinations of multiple state values respectively corresponding to state types. For example, as shown in FIGS. 6 and 7, if there are two types of states, a presence/absence of edema and SpO₂, the state is represented as a time series of combinations of categorical value of a presence/absence of edema and a value of SpO₂. Each of the patient's state assessment index and the doctor's state assessment index has one value for a state combination of each time in the time series of state combinations. The state assessment index map represents distribution of a patient's state assessment index and/or a doctor's state assessment index in a state space defined by multiple state types.

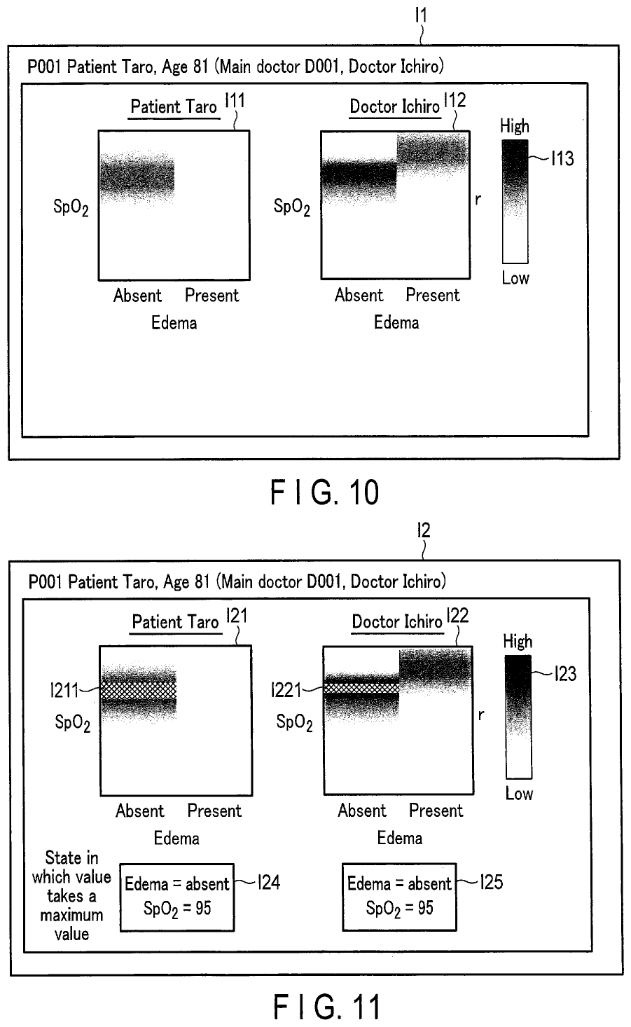

FIG. 10 is a diagram showing an example of the display screen I1 of a patient's state assessment index map I11 and a doctor's state assessment index map I12. In FIG. 10, the name of the patient is “Patient Taro” and the name of the doctor is “Doctor Ichiro”. As shown in FIG. 10, the display screen I1 displays a patient's state assessment index map I11 and a doctor's state assessment index map I12 side by side. The state assessment index maps I11 and I12 represent distribution of state assessment indices in a state space defined by a first state (presence/absence of edema) and a second state (SpO₂). One pixel in the state assessment index map I11 or I12 corresponds to one state combination. In the state assessment index maps I11 and I12, the horizontal axis indicates a categorical value (or discrete value) representing a presence/absence of edema, and the vertical axis indicates a continuous value of SpO₂ (e.g., values from 0 to 100). In the state assessment index maps I11 and I12, a value of a state assessment index is assigned to each combination of a presence/absence of edema and a continuous value of SpO₂. A value of the state assessment index is defined by a numerical value or an ordinal scale. A color value corresponding to a value of a state assessment index is assigned to a pixel of a state assessment index. Each pixel is displayed in a color corresponding to a color value assigned to the pixel. The correspondence between a value of a state assessment index and a color value is defined in the color table I13. It is preferable to display the color table I13 to allow users to know the correspondence between a value of a state assessment index and a color value.

The display of the state assessment index map I11 and I12 allows an observer to visually know the distribution of each of the patient's and doctor's state assessment indices. It is thus possible to know a desirability and sense of values of the patient and the doctor toward state combinations in a quantitative manner. Furthermore, displaying the state assessment index map I11 and the state assessment index map I12 side by side saves trouble and allows users to easily know a desirability and sense of values toward state combinations.

In the above example of the display, there are two types of states, a presence/absence of edema and SpO₂. However, the processing circuitry 21 displays a state assessment index in a case where there are three or more state types. If there are three or more state types, the processing circuitry 21 displays a state assessment index map relating to a patient and/or a state assessment index map relating to a doctor in a plane designated by one or two state types designated by an operator among those three or more state types. The one or two state types may be automatically set in accordance with a predetermined algorithm. Specifically, The one or two state types may be automatically determined on the basis of a contribution rate calculated for the state types.

As an example, if the state types are a presence/absence of edema, SpO₂, a therapeutic effect of BNP (brain natriuretic peptide), etc. and a medical bill, the state assessment indices are defined in a four-dimensional state space. Since it is difficult to display a distribution of state assessment indices in a four-dimensional state space as-is, an operator designates one or two state types as display targets, via the input device 23. The processing circuitry 21 sets a designated plane of the designated one or two state types as a state space, and displays the distribution of the state assessment indices in the designated plane. In other words, the state assessment index map I11, etc. in FIGS. 10 and 11, etc. can be called a distribution of state assessment indices in a designated plane defined by the presence/absence of edema and the SpO₂ value. In this case, it suffices that a discretionarily selected value is designated for a state type other than the state types targeted for display. If the display-targeted state types are a presence/absence of edema and SpO₂ in this specific case, it is necessary to set a value for BNP and a value for a medical bill in order to define a designated plane. The values for BNP and a medical bill may be designated discretionarily through the input device 23, etc., or automatically set in accordance with a predetermined algorithm.

After step S4, consensus building support according to the first embodiment is finished.

The method of displaying a patient's state assessment index and a doctor's state assessment index is not limited to the above-described method, and it is possible to modify or apply the method in various ways.

FIG. 11 is a diagram showing an example of the display screen I2 of a patient's state assessment index map I21 and a doctor's state assessment index map I22. The patient's state assessment index map I21 is the same as the patient's state assessment index map I11 shown in FIG. 10; the doctor's state assessment index map I22 is the same as the patient's state assessment index map I12 shown in FIG. 10; and the color table I23 is the same as that shown in FIG. 10.

As shown in FIG. 11, the processing circuitry 21 emphasizes a state combination I211 corresponding to a maximum value of the patient's state assessment index in the patient's state assessment index map I21, and a state combination I221 corresponding to a maximum value of the doctor's state assessment index in the doctor's state assessment index map I22. As an emphasis method, areas corresponding to the state combination I211 and the state combination I221 may be emphasized by colors, by flashing, or by drawing a box around them, for example. It is not necessary to emphasize both the state combination I211 and the state combination I221; only one of the state combination I211 and the state combination I221 may be emphasized. Emphasizing the state combination I211 and/or the state combination I221 allows an observer to easily know which state combination to pay attention to. An area to be emphasized is not necessarily an area corresponding to a maximum value of a state assessment index and may be an area corresponding to a state assessment index equal to or greater than a threshold. It suffices that the threshold value is set to a discretionarily determined value via the input device 23, etc.

As shown in FIG. 11, the processing circuitry 21 may display text information I24 of the state combination I211 and text information I25 of the state combination I221. For example, “edema=none”, “SpO₂=95”, etc. may be displayed as the text information I24 and the text information I25. This allows understanding of an actual meaning of the state combination I211 and the state combination I221.

As another example, the processing circuitry 21 may display a doctor's state assessment index in a first specific state in which the patient's state assessment index takes a predetermined value. As another example, the processing circuitry 21 may display a patient's state assessment index in a second specific state, wherein the doctor's state assessment index takes a predetermined value. The predetermined value may be set at a discretionarily selected statistic value, such as a maximum value or a minimum value, or a sum of state assessment indices equal to or greater than a threshold value or equal to or less than a threshold value.

As another example, the processing circuitry 21 may display a diagram showing changes over time of the state combination I211 corresponding to a predetermined value of a patient's state assessment index in the patient's state assessment index map I21 and/or the state combination I221 corresponding to a predetermined value of a doctor's state assessment index in the doctor's state assessment index map I22. The predetermined value may be set at a discretionarily selected statistic value, such as a maximum value or a minimum value, or a sum of state assessment indices equal to or greater than a threshold value or equal to or less than a threshold value.

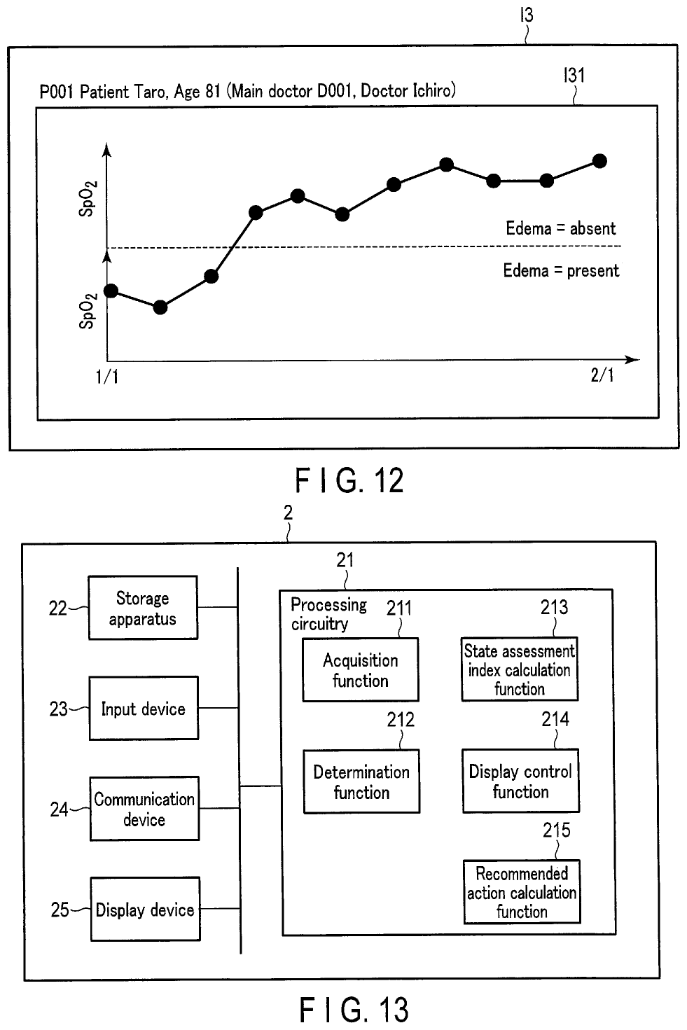

FIG. 12 is a diagram showing an example of the display screen I3 for change over time of a state combination corresponding to a maximum value of a state assessment index. As shown in FIG. 12, the display screen I3 displays the graph I31 of the change over time of the state combination corresponding to a maximum value of the patient's state assessment index. In the graph I31, the vertical axis indicates a presence/absence of edema and SpO₂, and the horizontal axis indicates a time (step). In the graph I31, a state combination that takes a maximum value of a patient's state assessment index in each time (for example, a date) is plotted. It is thereby possible for a user to know the change in a state combination over time when the maximum value is focused on. The change over time of a state combination corresponding to a maximum value of a doctor's state assessment index can also be displayed in a similar manner. Both of the change over time of the state combination corresponding to a maximum value of the patient's state assessment index and that of the doctor's state assessment index may be displayed side by side.

As described above, the consensus building support system 1 according to the present embodiment has a medical care/nursing care record storage apparatus 3 and a consensus building support apparatus 2. The medical care/nursing care record storage apparatus 3 stores a database that stores a state of a first user and actions by the first user and a second user. The consensus building support apparatus 2 determines whether an agent of the action was a first user or a second user. The consensus building support apparatus 2 calculates a first state assessment index representing a first user's assessment of the state based on a first user's state and an action relating to the first user, and calculates a second state assessment index representing a second user's assessment of the state based on the first user's state and an action relating to the second user. The consensus building support apparatus 2 causes a display device to display a first state assessment index and/or a second state assessment index.

According to the above configuration, it is possible to calculate first user's and second user's state assessment indices regarding a first user's state, without efforts required of the first user, etc. The display of the first user's and/or the second user's state assessment indices allows an observer to know the first user's and/or the second user's assessment of the first user's state in a quantitative manner. It is thus possible to achieve two-way shared decision making between the patient and the doctor. Thus, it is possible to easily know the preferences of multiple users in decision making on medical care.

In the above description, it was assumed that the first user is a patient and the second user is a doctor. However, the present embodiment is not limited to this example, and the first user and the second user may be the same person.

As an example, the first user and the second user may be the same patient. Actions taken in the past by a patient who suffers from a chronic disease and has been receiving long-term treatment can be compared with those in the present, so that changes in preferences over time may be revealed.

As another example, the first user and the second user may be the same doctor. It is possible to reveal changes in preferences over time by comparing the actions taken by the doctor in the past and those in the present. By revealing the changes in preferences, it is possible to bring consistency in treatment. It is also possible to check if the treatment is appropriately updated in line with changes in guidelines (for example, whether an administered treatment undesirably reverts to a treatment before the guidelines were changed).

As another example, the first user and the second user may be different doctors. It is possible to reveal differences in preferences between a first doctor and a second doctor by comparing the actions by the first and second doctors. For example, when a doctor in charge is changed due to relocation or other reasons, it is possible to check if the successor pursues the same policy for treatment as their predecessor did. As another example, if this technology is applied to a first doctor and a second doctor who work at different medical institutions, it is possible to check if a treatment is pursued on the same policy.

### Second Embodiment

Next, a consensus building support system, apparatus, and method according to the second embodiment are described. In the following explanation, structural elements having substantially the same functions as in the first embodiment will be denoted by the same reference symbols, and a repeat description will be given only where necessary.

FIG. 13 is a diagram showing a configuration example of a consensus building support apparatus 2 according to the second embodiment. As shown in FIG. 13, the processing circuitry 21 of the consensus building support apparatus 2 executes a program to implement recommended action calculation function 215, in addition to the acquisition function 211, the determination function 212, the state assessment index calculation function 213, and the display control function 214.

Through implementation of the display control function 214, the processing circuitry 21 display a difference between a first user's state assessment index and a second user's state assessment index. Through implementation of the recommended action calculation function 215, the processing circuitry 21 calculates a candidate for a recommended action for making the difference value smaller for a state combination in which the difference value between the first user's state evaluation index and the second user's state evaluation index is greater than a threshold value. The calculated candidate is displayed on the display device 25, etc. by implementation of the display control function 214.

An example of the operation of the consensus building support apparatus 2 according to the second embodiment is described below. In the following description, assume that a first user is a patient and a second user is a doctor, similarly to the explanation of the operation example in the first embodiment. Assume that the medical care/nursing care record storage apparatus 3 stores an electronic medical care record database for recording medical care records.

The processing circuitry 21 of the second embodiment display a difference value between a patient's state assessment index and a doctor's state assessment index. There are many ways of displaying a difference value. For example, the processing circuitry 21 may display a difference map representing distribution of differences between a patient's state assessment index and a doctor's state assessment index for respective state combinations. The difference may be an absolute value of a difference between a patient's state assessment index and a doctor's state assessment index, or a subtraction value obtained by subtracting a doctor's state assessment index from a patient's state assessment index, or a subtraction value obtained by subtracting a patient's state assessment index from a doctor's state assessment index. As another example, the processing circuitry 21 may display a difference value between a patient's state assessment index and a doctor's state assessment index in a first specific state in which the patient's state assessment index takes a predetermined value. Alternatively, the processing circuitry 21 may display a difference value between a patient's state assessment index and a doctor's state assessment index in a second specific state in which the doctor's state assessment index takes a predetermined value. The predetermined value may be set at a discretionarily selected statistic value, such as a maximum value or a minimum value, or a sum of state assessment indices equal to or greater than a threshold value or equal to or less than a threshold value.

As different examples of displaying a difference value, the processing circuitry 21 may display a graph representing change over time of a difference value between a patient's state assessment index and a doctor's state assessment index.

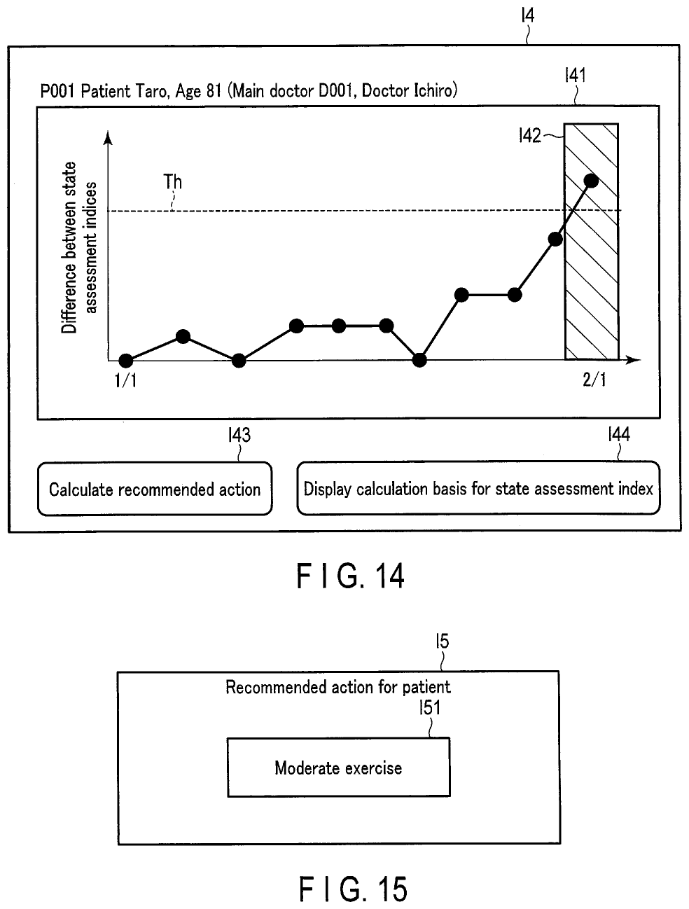

FIG. 14 is a diagram showing an example of a display screen I4 of a graph I41 representing a change over time of a difference between the patient's state assessment index and the doctor's state assessment index. As shown in FIG. 14, the graph I41 in which the vertical axis represents a difference value and the horizontal axis represents time (time and date) is displayed in the display screen I4. The difference may be an absolute value of a difference between a patient's state assessment index and a doctor's state assessment index, or a subtraction value obtained by subtracting a doctor's state assessment index from a patient's state assessment index, or a subtraction value obtained by subtracting a patient's state assessment index from a doctor's state assessment index. The display of the difference value allows an observer to know the disconnection between a patient's state assessment index and a doctor's state assessment index. The display of the change of difference value over time allows an observer to know the disconnection over time between a patient's state assessment index and a doctor's state assessment index.

As shown in FIG. 14, the graph I41 displays a threshold value Th for a difference value. The threshold value Th may be set based on any criteria; for example, the threshold value Th may be set in a boundary between an acceptable difference value and an unacceptable difference value. The processing circuitry 21 emphasizes a difference value equal to or greater than the threshold value Th with a frame line I42, etc. It is thereby possible to attract attention of the observer to a difference value equal to or greater than the threshold value Th.

As shown in FIG. 14, the display screen I4 displays the recommended action display button I43 and the calculation basis display button I44. When the recommended action display button I43 is pressed, the processing circuitry 21 calculates, through implementation of the recommended action calculation function 215, a candidate of a recommended action to compress a difference value equal to or greater than the threshold value Th. If there are two or more difference values that are equal to or greater than the threshold value Th, a difference value targeted for calculation may be designated via the input device 23, etc. The user targeted for the calculation of a recommended action candidate is selectable from a patient and/or a doctor via the input device 23, etc. The calculated recommended action candidate is displayed by the processing circuitry 21.

There are various methods for calculating a recommended action candidate. In one example, the processing circuitry 21 calculates a recommended action candidate by reinforcement learning based on a state and a state assessment index. Specifically, history data relating to various patients and doctors acquired in the past is recorded in the medical care/nursing care record storage apparatus 3. The components of the history of data of patients include a combination of a patient's state at a certain step, a patient's action corresponding to the state, a patient's state assessment index corresponding to the state, and a patient's state in a next step observed as a result of the action. The components of the history of data of doctors include a combination of a patient's state at a certain step, a doctor's action corresponding to the state, a doctor's state assessment index corresponding to the state, and a patient's state in a next step observed as a result of the action.

The processing circuitry 21 generates a trained model to which a patient's state of a step targeted processing and a difference value of state assessment indices of a user who is targeted for calculation, which are obtained from the history data of patients and doctors, are input and which outputs a recommended action candidate for making the difference value smaller for the user targeted for calculation. The trained model is stored in the storage apparatus 22. The processing circuitry 21 inputs a state of a patient of a step targeted for processing and a difference value of state assessment indices of a user targeted for calculation into the trained model and calculates a recommended action candidate. Alternatively, the processing circuitry 21 may specify a path of a patient's action with a highest state assessment index (reward) (an optimal action path) for the patient based on the history data and calculate a patient's action of the step targeted for processing in the optimal action path as a recommended action candidate.

The history data is not limited to actually obtained data of states, actions and state assessment indices relating to a patient and a doctor, and may be simulated data of states, actions and state assessment indices.

FIG. 15 is a diagram showing an example of a display window for a recommended action candidate. The display window I5 is superimposed on, for example, the display screen I4 displaying a difference value of the state assessment index shown in FIG. 14. The display window I5 includes a display section I51, and a character sequence indicating a recommended action is displayed in the display section I51. FIG. 15 shows “moderate exercise” as an example of a recommended action candidate for a patient on “February 1st” shown in FIG. 14.

When the calculation basis display button I44 shown in FIG. 14 is pressed down, the processing circuitry 21 displays a calculation basis of the recommended action candidate on a display.

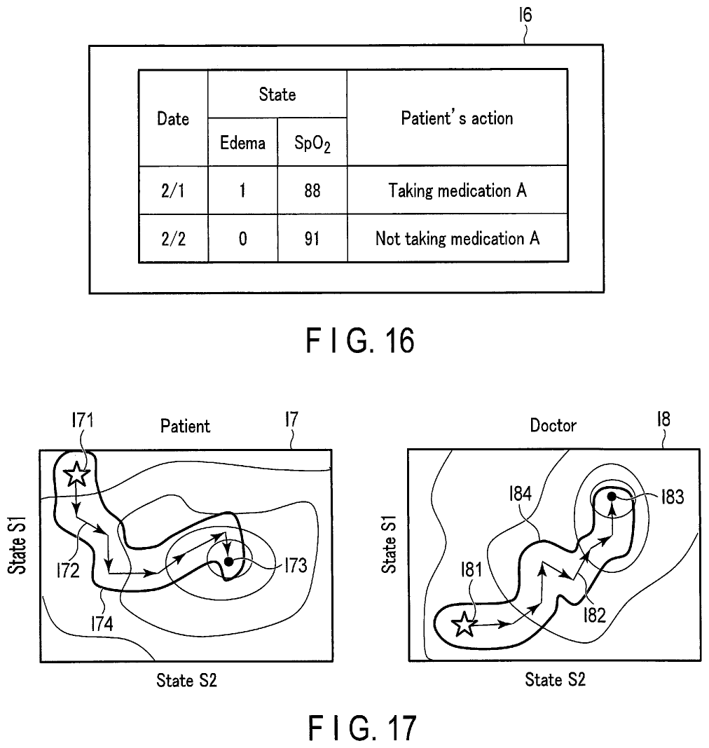

FIG. 16 is a diagram showing an example of a display window I6 of bases for calculating a recommended action candidate. As shown in FIG. 16, the display window I6 is superimposed on, for example, the display screen I4 of a difference between state assessment indices shown in FIG. 14. The display window I6 displays a state combination and an action for a date corresponding to the calculation-targeted difference value as a calculation candidate. For example, a recommended action candidate for a patient on February 1st, the state combination and the patient's action on February 1st, which is the calculation-targeted step, and the state combination and the patient's action of February 2nd, which is the next step, are displayed.

In the foregoing example, the difference value between the patient's state assessment index and the doctor's state assessment index was assumed to be all state combinations, in other words, a sum of difference values between the patient's state assessment index and the doctor's state assessment index over the entire state space. However, the method of calculating a difference value is not limited to this example, and various methods are possible.

As an example, the processing circuitry 21 may calculate a difference value between the patient's state assessment index and the doctor's state assessment index, limiting the targeted area in the state space. Specifically, the processing circuitry 21 may calculate a difference value between the patient's state assessment index and the doctor's state assessment index by limiting the state space to a peripheral area of a user's optimal action path (hereinafter, a “path peripheral area”).

FIG. 17 is diagrams showing a path peripheral area I74 of an optimal action path I72 of a patient and a path peripheral area I84 of an optimal action path I82 of a doctor. The left diagram in FIG. 17 schematically shows the path peripheral area I74 of the patient's optimal action path I72 in the state space I7, and the right diagram in FIG. 17 schematically shows the path peripheral area I84 of the doctor's optimal action path I82 in the state space I8. As shown in FIG. 17, the state spaces I7 and I8 are a two-dimensional state space represented by a state S1 and a state S2. As shown in the left diagram of FIG. 17, an optimal action path I72 relating to a patient connecting an initial state I71 and a final state I73 in the state space I7 is determined. The optimal action path I72 means a path of a patient's action with a highest reward in the state space I7. Similarly, as shown in the right diagram of FIG. 17, an optimal action path I82 relating to a doctor and connecting an initial state I81 with a final state I83 in the state space I8 is determined. The optimal action path I82 is a path of a doctor's action with a highest reward in the state space I8. The optimal action paths I72 and I82 that are expanded by a predetermined distance are set as the path peripheral areas I74 and I84. The predetermined distance may be set to a discretionarily determined value.

The processing circuitry 21 calculates a first sum value of state assessment indices of all state combinations included in the path peripheral area I74 and a second sum value of state assessment indices of all state combinations included in the path peripheral area I84, and calculates a difference value between the first sum value and the second sum value as a state assessment index difference value. Specifically, the processing circuitry 21 calculates a value obtained by subtracting the first sum value from the second sum value or a value obtained by subtracting the second sum value from the first sum value as a state assessment index difference value. The calculated state assessment index difference value is displayed on the display device 25 by the processing circuitry 21. At this time, the processing circuitry 21 may display the state assessment index difference value, together with the optimal action paths I72 and I82 the path peripheral areas I74 and I84, and the state spaces I7 and I8 shown in FIG. 17. It is thereby possible for an observer to know the basis for the state assessment index difference value.

As another example, the processing circuitry 21 may map the path peripheral area I74 to the state space I8 and calculate a difference value between a sum of state assessment indices of all state combinations included in this mapped area and a first sum value as a state assessment index difference value. Similarly, the processing circuitry 21 may map the path peripheral area I84 to the state space I7 and calculate a difference value between a sum of state assessment indices of all state combinations included in this mapped area and a second sum value as a state assessment index difference value.

In the above description, it was assumed that the difference value between the sum values of the state assessment indices are calculated as a state assessment index difference value; however, a difference value between average values of the state assessment indices may be calculated as a state assessment index difference value.

As another example, the processing circuitry 21 may calculate a state assessment index difference value for each of the past action path and the present action path if the current state has not reached a final state.

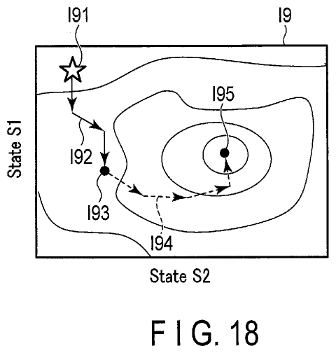

FIG. 18 is a diagram showing an example of a past action path I92 and a future action path I94 in the state space I9. A past action path I92 and a future action path I94 may be either a patient's action path or a doctor's action path. The past action path I92 is an action path actually taken by a user between the initial state I91 to the current state I93. The future action path I94 is an optimal action path that can be taken between the current state I93 and the final state I95. It suffices that the future action path I94 is calculated by reinforcement learning based on the past action path I92, each state between the initial state I91 and the current state I93, and the state assessment index.

As an example, the processing circuitry 21 calculates a sum value of state combinations over the past action path I92 and a sum value of state combinations of the future action path I94, and displays the same. At this time, the processing circuitry 21 may display the past action path I92 and the future action path I94, such as those shown in FIG. 18, together with a sum value relating to the past action path I92 and a sum value relating to the future action path I94. The processing circuitry 21 may display either one of a sum value relating to the past action path I92 and a sum value relating to the future action path I94.

As another example, the processing circuitry 21 may specify an area in which a state assessment index takes a predetermined value in a patient's state space, map the area on a doctor's state space, and calculate a difference value between a value of the state assessment index in this mapping area (doctor's state assessment index value) and a patient's state assessment index predetermined value as the state assessment index difference value. The predetermined value may be set at either a maximum value of a state assessment index or a value equal to or greater than a threshold value. Similarly, the processing circuitry 21 may specify an area in which a state assessment index takes a predetermined value in a doctor's state space, map the area on a patient's state space, and calculate a difference value between a value of the state assessment index in this mapping area (patient's state assessment index value) and a patient's state assessment index predetermined value as the state assessment index difference value. The area in which a state assessment index takes a predetermined value is not limited to an area that coincides with a state combination in which the state assessment index takes a predetermined value and may be an area of a predetermined size, such as 3×3 or 5×5, including a state combination in which the state assessment index takes a predetermined value.

As another example, the processing circuitry 21 may calculate a state assessment index difference value, limiting a target area to an action path of various patients. Specifically, the processing circuitry 21 acquires a plurality of past action paths relating to a plurality of patients from the history data, and specifies a typical action path of the patients in the past (hereinafter, a “typical path”) based on the acquired action paths. A typical path may be set to an action path passed by a threshold number or more action paths. Then, the processing circuitry 21 calculates a difference value between the patient's and doctor's state assessment indices over the typical path included in the state space. By limiting the target to the typical path, it is possible to compare the patient's state assessment index and the doctor's state assessment index on a unified action path, regardless of individual patients.

According to at least one of the foregoing embodiments, it is possible to easily know preferences in the shared decision making relating to medical care and/or nursing care between users.

The term “processor” used in the above explanation indicates, for example, a circuit, such as a CPU, a GPU, or an Application Specific Integrated Circuit (ASIC), and a programmable logic device (for example, a Simple Programmable Logic Device (SPLD), a Complex Programmable Logic Device (CPLD), and a Field Programmable Gate Array (FPGA)). The processor realizes its function by reading and executing the program stored in the storage circuitry. The program may be directly incorporated into the circuit of the processor instead of being stored in the storage circuit. In this case, the processor implements the function by reading and executing the program incorporated into the circuit. The function corresponding to the program may be implemented by a combination of logic circuits instead of executing the program. Each processor of the present embodiment is not limited to a case where each processor is configured as a single circuit; a plurality of independent circuits may be combined into one processor to realize the function of the processor. Furthermore, a plurality of constituent elements shown in FIGS. 1, 2, and 13 may be integrated into one processor to implement the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A consensus building support system comprising:
a storage apparatus that stores a database storing a state of a first user and actions taken by the first user and a second user; and
processing circuitry configured to:
calculate a first state assessment index representing a first user's assessment of the state based on the first user's state and the actions relating to the first user, and calculate a second state assessment index representing a second user's assessment of the state based on the first user's state and the actions relating to the second user; and
display the first state assessment index and/or the second state assessment index on a display device.

2. The consensus building support system according to claim 1, wherein
the processing circuitry is configured to determine whether an agent of the action is the first user or the second user.

3. The consensus building support system according to claim 2, wherein
if the action is an action acquired from an instruction, the processing circuitry determines that an agent of the action is a doctor who is the second user, and if the action is an action acquired from an administration order, the processing circuitry determines that an agent of the action is a patient who is the first user.

4. The consensus building support system according to claim 2, wherein
if the action is an action acquired from the “P” section of a SOAP note, the processing circuitry determines that an agent of the action is a doctor who is the second user, and if the action is an action acquired from the “S” section of a SOAP note, the processing circuitry determines that an agent of the action is a patient who is the first user.

5. The consensus building support system according to claim 2, wherein
if the action is an action acquired from a predetermined item included in an order, the processing circuitry is configured to determine whether the agent of the action is a doctor who is the second user or a patient who is the first user in accordance with an identifier assigned to the predetermined item.

6. The consensus building support system according to claim 2, wherein
the processing circuitry performs natural language processing on a character sequence that includes characters describing the action to determine whether an agent of the action is a doctor who is the second user or a patient who is the first user.

7. The consensus building support system according to claim 1, wherein
the processing circuitry is configured to:
calculate the first state assessment index by inverse reinforcement learning based on a state of a processing-targeted step and a state of a step next to the processing-targeted step among the acquired states, and an action of the first user taken between the state of the processing-targeted step and the state of the next step; and
calculate the second state assessment index by inverse reinforcement learning based on a state of a processing-targeted step and a state of a step next to the processing-targeted step among the acquired states, and an action of the second user taken between the state of the processing-targeted step and the state of the next step.

8. The consensus building support system according to claim 1, wherein
the state includes a time series of combinations of a plurality of state values respectively corresponding to a plurality of state types,
each of the first state assessment index and the second state assessment index has one value for the combination of each time in the time series.

9. The consensus building support system according to claim 8, wherein
the processing circuitry is configured to display distribution of the first state assessment index and/or the second state assessment index in a state space defined by the plurality of state types on a display.

10. The consensus building support system according to claim 9, wherein
if there are three or more state types, the processing circuitry display distribution of the first state assessment index and/or the second state assessment index in a designated plane defined by one or two state types designated by an operator among the plurality of state types on a display.

11. The consensus building support system according to claim 9, wherein
the processing circuitry emphasizes in the distribution a first specific state in which the first state evaluation index takes a predetermined value and/or a second specific state in which the second state evaluation index takes a predetermined value.

12. The consensus building support system according to claim 11, wherein
the processing circuitry is configured to display a diagram showing change over time of the first specific state and/or the second specific state on a display.

13. The consensus building support system according to claim 1, wherein
the processing circuitry displays a difference value between the first state assessment index and the second state assessment index on a display.

14. The consensus building support system according to claim 13, wherein
the processing circuitry displays a graph indicating change over time of a difference value between the first state assessment index and the second state assessment index on a display.

15. The consensus building support system according to claim 14, wherein
if the difference value has a value equal to or greater than a threshold value, the processing circuitry emphasizes a time corresponding to a value equal to or greater than the threshold value in the graph.

16. The consensus building support system according to claim 13, wherein
the processing circuitry is configured to:
calculate a candidate of a recommended action for making the difference smaller by reinforcement learning based on the state and actions; and
display the candidate on the display.

17. The consensus building support system according to claim 1, wherein
the processing circuitry is configured to:
display the second state assessment index in a first specific state in which the first state evaluation index takes a predetermined value on a display;
display a difference value between the first state assessment index and the second state assessment index in the first specific state on the display;
display the first state assessment index in a second specific state in which the second state evaluation index takes a predetermined value on the display; or
display a difference value between the first state assessment index and the second state assessment index in the first specific state on the display.

18. The consensus building support system according to claim 1, wherein
the first user is a patient, and
the second user is family of the patient, a clinician and/or a nursing care service worker.

19. A consensus building support apparatus comprising:
processing circuitry configured to:
acquire a state of a first user and actions of the first user and a second user from a database;
calculate, based on the first user's state and the actions relating to the first user, a first state assessment index representing a first user's assessment of the state based on the first user's state and the actions relating to the first user, and calculate, based on the first user's state and the actions relating to the second user, a second state assessment index representing a second user's assessment of the state; and
display the first state assessment index and/or the second state assessment index on a display.

20. A consensus building support method comprising:
acquiring a state of a first user and actions of the first user and a second user from a database;
calculating, based on the first user's state and the actions relating to the first user, a first state assessment index representing a first user's assessment of the state based on the first user's state and the actions relating to the first user, and calculate, based on the first user's state and the actions relating to the second user, a second state assessment index representing a second user's assessment of the state; and
display the first state evaluation index and/or the second state evaluation index on a display.
